(19) United States
(12) Patent Application Publication
(10) Pub. No.: US 2020/0281889 A1
(43) Pub. Date: Sep. 10, 2020

(54) Edible Film Comprising Adjacent Conjoined Strips

(71) Applicant: Terpene Therapeutics Inc., Ottawa (CA)

(72) Inventor: Michael Bousfield, Ottawa (CA)

(21) Appl. No.: 16/295,554

(22) Filed: Mar. 7, 2019

(51) Int. Cl. A61K 31/352; A61K 9/00; A61K 36/185; A61K 31/05; A61K 47/38; A61K 47/10; A61K 47/32; A61K 47/20; A61K 47/12

(52) U.S. Cl. CPC A61K 31/352; A61K 9/006; A61K 36/185; A61K 31/05; A61K 45/06; A61K 47/10; A61K 47/32; A61K 47/20; A61K 47/12; A61K 47/38

(57) ABSTRACT

Edible terpene-containing films are provided comprising at least one terpene or terpenoid, at least one film-forming agent, at least one surfactant or emulsifier, and a plasticizer. The films may be coated with a buccal and/or sublingual mucosal absorption enhancer, an anti-microbial agent and/or comprise at least two adjacent conjoined strips having a distinct functionalities.

### FIELD OF THE INVENTION

The present invention generally relates to edible films, and more particularly relates to edible films comprising adjacent adjoined or conjoined strips.

### BACKGROUND OF THE INVENTION

Edible film products are known in the art. They were traditionally provided in simple strip form, but have been modified over the years to assume, for example, different shapes, layers, colours and/or flavours.

Edible oral care strips that provide a breath-freshening function have been in existence for some time, and typically comprise a film-forming agent and a flavor agent such as menthol, peppermint or spearmint.

Novel forms of consumable edible thin films have more recently been developed as described, for example, in US Patent Application No. 2004/0096569 to Barkalow et al. that vary in color, flavor, shape and mouthfeel, to improve the marketability of edible films to younger consumers.

US 2003/0224090 describes edible films as snacks devised to provide flavor and/or craving satisfaction. These films comprise high levels of appealing flavors and flavor combinations, as well as sweeteners and other ingredients. The snack may include one or more layers to preserve structure, or to enhance flavor sensation.

### SUMMARY OF THE INVENTION

Novel edible films are herein provided comprising adjacent adjoined or conjoined strips.

Thus, in one aspect of the invention, an edible film is provided comprising at least one terpene or terpenoid, at least one film-forming agent, a surfactant and/or emulsifier and a plasticizer, wherein said film comprises at least two adjacent conjoined strips, each strip having a distinct functionality.

In another aspect of the invention, an edible film is provided comprising at least one terpene or terpenoid, at least one film-forming agent, a surfactant and/or emulsifier and a plasticizer, wherein the film is coated with a buccal and/or sublingual mucosal absorption enhancer and/or an anti-microbial agent.

These and other aspects of the invention are described by reference to the following figures.

### BRIEF DESCRIPTION OF THE FIGURES

FIG. 1 illustrates the terpene profile of the Blue Dream strain;

FIG. 2 illustrates the terpene profile of the Granddaddy Purple strain;

FIG. 3 illustrates the terpene profile of the Girl Scout Cookies strain;

FIG. 4 illustrates the terpene profile of the Green Crack strain;

FIG. 5 illustrates the terpene profile of the OG Kush;

FIG. 6 illustrates the terpene profile of the Sour Diesel strain; and

FIG. 7 illustrates properties of terpenes of different classes.

### DETAILED DESCRIPTION OF THE INVENTION

An edible film is provided comprising at least one terpene or terpenoid, at least one film-forming agent, at least one surfactant or emulsifier, and a plasticizer, wherein the film comprises at least two adjacent adjoined or conjoined strips, each strip having a distinct functionality.

The film comprises at least two adjacent adjoined or conjoined strips having distinct functionalities. The term “adjoined” or “conjoined” as it relates to adjacent strips refers to strips which are joined together or contiguous. The term “functionality” is used herein to refer to the utility or function of a strip. Functionality may include dissolution properties, delivery properties, adhesion properties, therapeutic properties, flavor, etc. The functionality of the strips of a film may relate to the same property or different properties. For example, a film may be provided with two or more conjoined strips in which each strip exhibits a different dissolution rate, in which each strip exhibits different adhesion properties, or in which each strip has different doses of the same therapeutic with different dissolution rates. Alternatively, a film may be provided with two or more conjoined strips having unrelated functionalities, for example, a film with two or more conjoined strips having different therapeutic utility, or a film with a flavor strip and a therapeutic strip which may have the same or different dissolution rates.

The film may comprise any edible film forming agent including, but not limited to, pullulan, sodium alginate, carrageenan, methyl cellulose, ethyl cellulose, hydroxypropylmethyl cellulose, hydroxypropyl cellulose, corn starch, pectin, chitin, chitosan, collagen, gelatin, zein, gluten, cassava starch, glucan and mixtures thereof.

The film forming agent may be used alone or combined with one or more agents that facilitate film forming, including but not limited to, one or more of hydroxyethyl cellulose, polyvinyl pyrrolidone, carboxymethyl cellulose, polyvinyl alcohol, polyethylene glycol, polyacrylamides, glycolide, polylactic acid, polylactide, cellulose gum, xanthan gum, tragacanth gum, guar gum, acacia gum, arabic gum, gum karaya, welan gum, polyacrylic acid, sodium methacrylic acid polymers, methacrylic acid copolymers, acrylic acid polymers, acrylic acid copolymers, methylmethacrylate copolymer, carboxyvinyl polymer, amylose, high amylose starch, casein, algin, maize, chondrus crispus, crosspovidone (CPV), polyvinylpyrrolidone (PVP), hydroxymethylcellulose, glycogen, phytoglycogen, polysaccharides, hydroxypropylate, dextrin, maltodextrin, soy protein isolate, whey protein isolate, beeswax, carnauba wax, fungal exopolysaccharides, rapeseed, and mixtures thereof.

The film forming agent, or combination of film-forming agents, is present in an amount ranging from about 0.01 to about 99 wt %, preferably from about 30 to about 80 wt %, more preferably from about 45 to about 75 wt % of the film, and even more preferably, from about 60 to about 65 wt % of the film. A preferred film forming agent is pullulan.

In one embodiment, the present film may comprise one or more fast-dissolving strips in which the film forming agent is selected from pullulan, sodium alginate, and mixtures thereof to result in a film that dissolves in 5-60 seconds as measured using a dissolve rate test such as a permeation test that mimics the oral environment. In an example, quick dissolving film strips comprise pullulan in an amount ranging from 1 to 99%, optionally combined with sodium alginate, in an amount ranging from about 1 to 99% is provided. The strips may exhibit different dissolution rates by altering the film forming agent content of the strips. For example, by using a fast dissolving film forming agent, or a greater content of a fast dissolving film forming agent such as pullulan, the dissolution rate of the film can be increased, while using a film forming agent that dissolves at a slower rate, e.g. alginate, or a greater content of such a film forming agent, the dissolution rate of the film is decreased. Dissolution may also be altered by adjusting the pH of the film, for example, increasing the pH of the film, will generally increase dissolution rate of the film.

In another embodiment, the present film may comprise one or more slow dissolving strips in which the film forming agent is selected from hydroxypropyl methylcellulose, carrageen, and mixtures thereof, additionally comprising one or more facilitating agents such as maltodextrin to result in a film that dissolves in 61-180 seconds according to a permeation test. By altering the film forming agent content of a strip, different dissolution rates may be attained. For example, a slow dissolving film may be attained which comprises hydroxypropyl methylcellulose, carrageen and maltodextrin in amounts ranging from 11-45% by wt HPMC, 1-23% by wt carrageen and 5-29% by wt maltodextrin. Higher amounts of HPMC would result in a slower dissolving strip, while higher amounts of sodium alginate would result in a less slow dissolving strip.

Any suitable method may be used to determine dissolve rate of a film. An exemplary method includes tautly fastening a sample of the film (preferably, 30 to 35 mm long, 20-25 mm in width and a thickness of 0.053 mm-0.063 mm) with adhesive tape on the test plate of a dissolution testing apparatus. A chrome ball bearing (about 6.35 mm in diameter, and 1.045+/−0.002 g) is placed centrally on top of the film and 2 drops of water at ambient temperature are dropped onto the top of the ball bearing. The time for the ball to drop to the test plate 10 mm below represents the dissolution rate. The longer the time, the slower the dissolution rate. This method is referred to herein as EI QC 019.

A film may be provided comprising one or more fast-dissolving strips as well as one or more slow dissolving strips. Thus, the film may comprise a strip or strips comprising pullulan, sodium alginate, or mixtures thereof to form a fast-dissolving strip, and a strip or strips comprising hydroxypropyl methylcellulose, carrageen, or mixtures thereof to form a slower-dissolving strip or a strip that dissolves at a reduced rate. The provision of a film comprising strips that dissolve at different rates permits staged delivery of the terpene content of the film, as well as any other active ingredients present therein. For example, terpene or other active ingredient may be contained in a fast-dissolving strip to promote increased mucosal uptake thereof, and reduce the amount that bypasses mucosal uptake. Terpene or other active ingredient may be contained in a slower-dissolving strip in order to allow a buccal absorption enhancer to have an effect on the buccal membrane before release of the active ingredient, thereby resulting in increased absorption of the active ingredient on its release from the film.

The present film will generally also include a surfactant, i.e. a compound that lowers the surface tension (or interfacial tension) between two liquids or between a liquid and a solid. Examples of surfactants include, but not limited to, glycerol, glycerin, propylene glycol, sucrose fatty acid ester, sorbitan monostearate, sorbitol-based surfactants such as polysorbate 60 or 80, lecithin such as soy or sunflower lecithin, modified cellulose gum (CMC), ceratonia gum (locust bean gum), sodium lauryl sulfate (SLS), or mixtures thereof. The film will generally comprise a surfactant in an amount ranging from about 0.5 to 15% by wt, e.g. 0.5 to 12.7 wt %.

An emulsifier is a type of surfactant, and functions to stabilize an emulsion. The film may include an emulsifier in an amount of about 1% to 15% by wt. Examples of suitable emulsifiers include but are not limited to polyoxyethylene, sorbitan monostearate, lecithin such as soy lecithin, stearate salt such as potassium stearate, calcium stearate or sodium stearate, calcium stearoyl lactylate (CSL), glyceryl monostearate, mono propylene glycol, sorbitan monooleate (SPAN 80), sorbitol-based emulsifiers such as polysorbate 60 or 80 (Tween 60/80), monoglyceride, glycerol triacetate and mixtures thereof.

The film may include a surfactant as well as an emulsifier, for example, sorbitol-based surfactants, such as polysorbate 60 or 80, glycerin or propylene glycol, combined with emulsifiers such as sorbitan monostearate, a stearate salt, or polyoxyethylene.

The film will generally also include plasticizer that increases the plasticity and/or decreases the viscosity of the film. Examples of plasticizers include, but are not limited to, monostearate palm oil, lethicin, sorbitan monostearate, glycerin, glycerin fatty acid ester, triacetin, pre-gelatinized starch, sorbitol, medium chain mono- or diglycerides, castor oil, malic acid, polyethylene glycol (PEG), glycerol, palm oil and mixtures thereof. The film will generally comprise plasticizer in an amount ranging from about 2 to 18 wt %.

It is noted that some compounds may have multiple functions, and thus, satisfy multiple roles in the present film, for example, sorbitol-based compounds function as both a surfactant and an emulsifier, sorbitan monostearate and lecithin function as a surfactant, emulsifier and plasticizer, and glycerol functions as both a surfactant and a plasticizer.

The film may include a sweetener in an amount in the range of 0% to 11% by wt. Examples of suitable sweeteners include but are not limited to: sucralose, acesulfame potassium, aspartame, stevia/rebaudioside, xylitol, neotame, saccharin, erythritol, isomalt, maltitol, lactitol, mannitol, glycerol (glycerine), glycyrrhizin and mixtures thereof.

The film may include a flavouring agent in an amount ranging from 0.1% to 15% by wt of the film, Examples of flavours include, but are not limited to, peppermint, strawberry, cherry, menthol, vanillin, fenugreek seed extract, spearmint, raspberry, ginger, pomegranate, passion fruit, peach, orange, tutti fritti, cola, honeydew, yuzu, cinnamon, mango, kumquat, lychee, grapefruit, spice extracts, and terpenes, e.g. limonene, myrcene, etc.

The present film comprises at least one terpene or terpenoid in an amount ranging from about 0.01% to 30% by wt. The term “terpene” or “terpenoid” refers to hydrocarbons derived from units of isopentenyl pyrophosphate and to functionally equivalent derivatives thereof. Examples of terpenes include myrcene, beta caryophyllene, pinene, limonene, terpinolene, humulene, nerolidol, linalool, ocimene, guaiol, bisabolol, alpha phellandrene, cadinene, camphene, camphor, citral, citronellol, delta 3-carene, eucalyptol, eugenol, gamma terpinene, geraniol, neral, ocimene, paracymene, phytol, pulegone, terpineol, valencene, functionally equivalent analogues or derivatives thereof such as oxides thereof. The term “functionally equivalent” as it relates to analogs and derivatives of a terpene refers to compounds which exhibit the same or similar activity or effect, e.g. at least about 50% of the activity of the terpene from which it is derived.

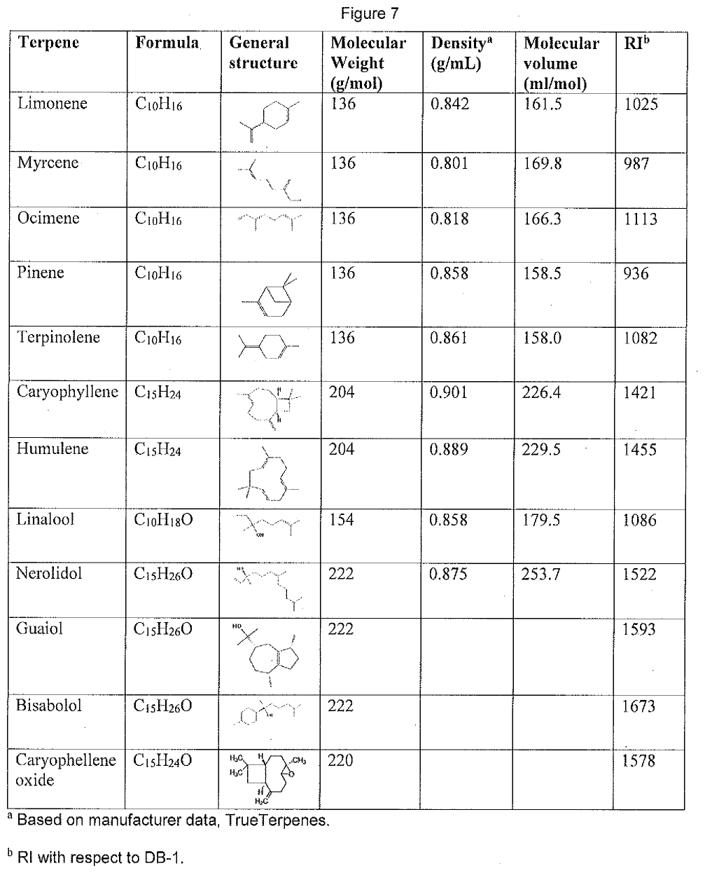

Terpenes may be classified by the number of molecular isoprene units. Thus, terpenes herein include hemiterpenes having a single isoprene unit such as isoprene and oxygen-containing derivatives such as prenol and isovaleric acid; monoterpenes having two isoprene units and the molecular formula C₁₀H₁₆ such as geraniol, terpineol, limonene, myrcene, ocimene, terpinolene, pinene and iridoids which are derived from monoterpenes; sesquiterpenes having three isoprene units and the molecular formula C₁₅H₂₄, such as humulene, caryophyllene, farnesenes, farnesol, B-bisabolene, B-curcimene, lanceol, juvabione and humbertiol; diterpenes having four isoprene units and the molecular formula C₂₀H₃₂. derive from geranylgeranyl pyrophosphate and include cafestol, kahweol, cembrene and taxadiene (precursor of taxol); sesterterpenes having 25 carbons and five isoprene units, such as geranylfarnesol; triterpenes having six isoprene units and the molecular formula C₃₀H₄₈ such as squalene; sesquarterpenes having seven isoprene units and the molecular formula C₃₅H₅₆, such as ferrugicadiol and tetraprenylcurcumene; and tetraterpenes having eight isoprene units and the molecular formula, C₄₀H₆₄, such as acyclic lycopene, the monocyclic gamma-carotene, and the bicyclic alpha- and beta-carotenes. Terpenes may also be classified as terpene derivatives or analogues such as hydroxylated terpenes such as linalool, nerolidol, guaiol and bisabolol; and oxygenated terpenes such as caryophyllene oxide and limonene oxide. The properties of some of the terpenes within these classes are shown in FIG. 7. Preferential terpene release can be fine-tuned based on differences or similarities thereof, such as molecular volume and retention index (RI).

The film may also include one or more additional active ingredients in an amount in the range of about 0.01% to 15% by wt, such as nutraceutical, pharmaceutical or therapeutic agents, including, but not limited to, botanical and/or herbal extracts, vitamins and minerals, pain medication (such as acetaminophen, non-steroidal anti-inflammatory agents (NSAIDS) such as aspirin, ibuprofen and diclofenac, anti-bacterial agents, decongestants, antihistamines, psychological treatments (e.g. anti-depressive and anti-anxiety agents), gastrointestinal treatments, sleep disorder treatments, cannabinoids, and mixtures thereof.

The term “cannabinoids” is meant to encompass phytocannabinoids, endocannabinoids (such as arachidonoyl-ethanolamide (anandamide), 2-arachidonoyl glycerol (2-AG) and arachidonyl glyceryl ether (noladin ether)), synthetic cannabinoids (manufactured artificially), and functionally equivalent derivatives and analogues of any of these. Examples of cannabinoids include, but are not limited to, cannabidiol (CBD), cannabidiol acid (CBDA), cannabinol (CBN), cannabigerol (CBG), cannabigerol acid (CBGA), cannabidivarin (CBDV), cannabidivarin acid (CBDVA), cannabinovarin (CBNV), cannabigerovarin (CBGV), cannabichromene (CBC), CBCV (cannabichromevarin), naphthoylindoles such as JWH-018, JWH-073, JWH-398, JWH-200, JWH-081, 4-methyl-JWH-073, JWH-015, JWH-122, JWH-220, JWH-019, JWH-007; phenylacetylindoles such as JWH-250 and JWH-203; benzoylindoles such as RCS-4, AM-694 and WIN 48,098; cyclohexylphenoles such as CP 47,497-C8 and CP 47,497; and HU-210. Cannabinoids also include tetrahydrocannabinoid and analogs thereof, namely, delta-9 tetrahydrocannabinol (THC) and functionally equivalent compounds, including analogs and derivatives thereof such as delta-8 tetrahydrocannabinol (D8-THC), tetrahydrocannabinol acid (THCA), tetrahydrocannabivarin (THCV), tetrahydrocannabivarin acid (THCVA), nabilone, rimonabant (SR141716), JWH-018, JWH-073, CP-55940, dimethylheptylpyran, HU-210, HU-331, SR144528, WIN 55,212-2, JWH-133, levonantradol and AM-2201. The term “functionally equivalent” as it relates to analogs and derivatives of a cannabinoid refers to compounds which exhibit the same or similar therapeutic effect, e.g. at least about 50% of the activity of the cannabinoid from which it is derived. Cannabinoids may be extracted from the cannabis plant using methods well-established in the art. Many of the cannabinoids may also be prepared using standard chemical synthetic methods. Some of these compounds are also commercially available.

The film may additionally contain a mucosal permeation or absorption enhancer, e.g. sublingual and/or buccal permeation enhancer, to aid in delivery of the terpene and any other active ingredients. An absorption enhancer in an amount in the range of about 0.01 to 5% by wt may be included in the film. Examples of absorption enhancers that may be used include, but are not limited to, 23-lauryl ether, aprotinin, azone, taurine, dextran sulfate, cetylpyridinium chloride, lauric acid, menthol, xylitol, oleic acid, polyoxyethylene, polysorbate 80, salts of EDTA, deoxycholate, glycocholate, lauryl sulfate, glycodeoxycholate, salicylate, taurocholate and dodecyl sulphate (e.g. sodium salts), palmitoyl carnitine and alkyl glycosides. Terpene absorption enhancers may also be used including alpha-limonene, dextro-limonene, nerolidol, menthol, alpha-bisabolol and eucalyptol.

An anti-oxidant may also be included in the film to prevent or reduce oxidation of the active ingredients in the film. Examples of suitable anti-oxidants include, but are not limited to, sodium thiosulfate, ascorbic acid, fatty acid esters of ascorbic acid, alpha-tocopherol, propyl gallate, erythorbic acid, sodium erythorbate, citric acid, tocopherols and alkyl-substituted paraben or salts thereof such as ethyl paraben, methyl paraben, propyl paraben or sodium salts thereof. Antioxidant may be included in the present film in an amount suitable to reduce or prevent oxidation, for example, an amount in the range of about 0.01 to 2.5% by wt.

In one embodiment, the adjacent conjoined strips of the present film each comprise a different terpene or combination of terpenes, or may contain in one strip a terpene or terpenes and one or more pharmaceutical, nutraceutical or therapeutic agents in adjacent strips in amounts (dosages) sufficient for their function.

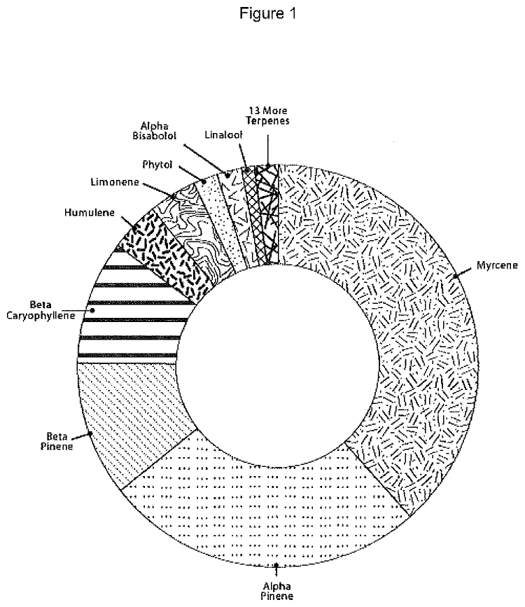

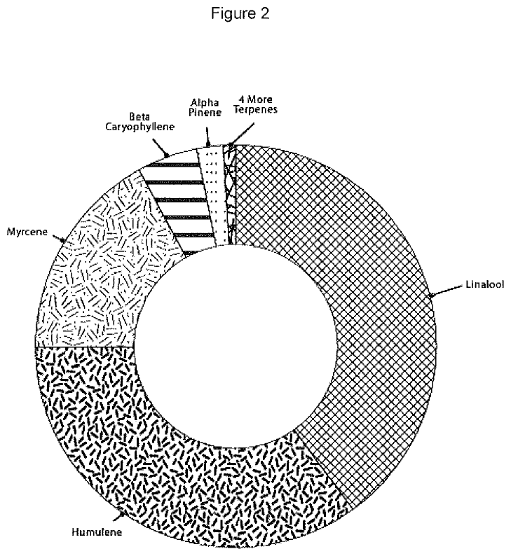

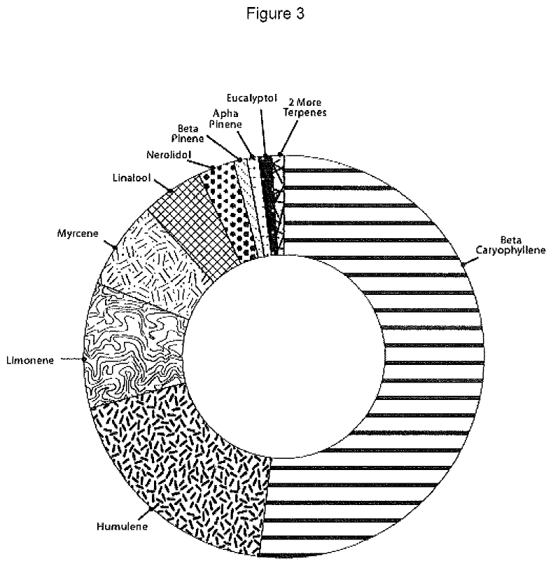

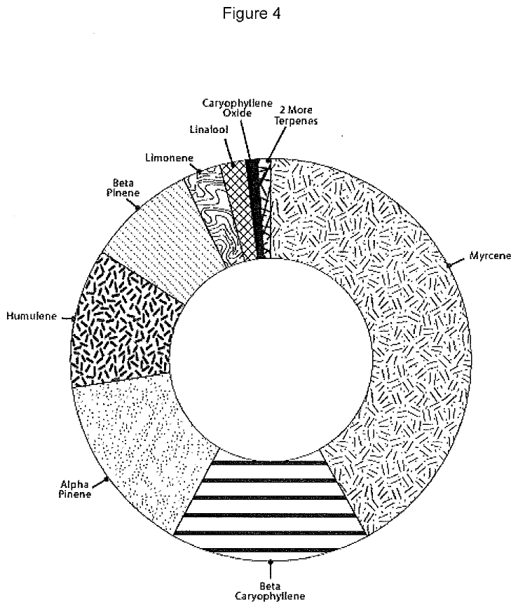

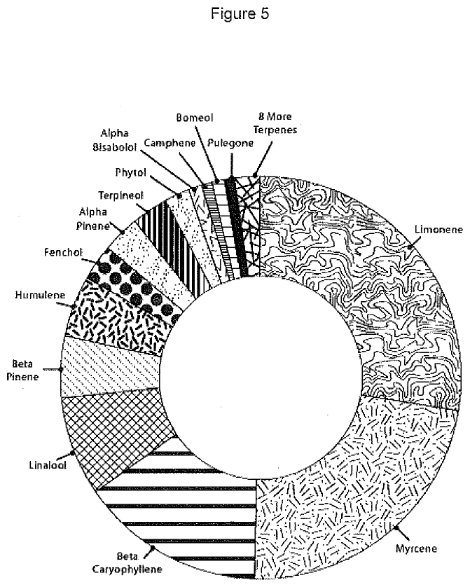

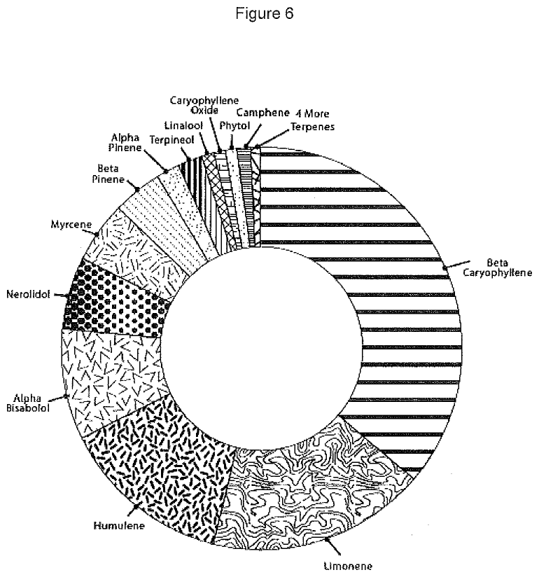

For example, the present film may include two or more strips that mimic the terpene content or profile, and optionally the cannabinoid content, of naturally occurring Cannabis strains such as Blue Dream, Bubba Kush, Granddaddy Purple, Girl Scout Cookies, Green Crack, Jack Herer, OG Kush, Sour Diesel, White Widow, GG #4, SFV OG, Lemon Skunk, Gelato, Strawberry Banana, Orange Cookie, Cherry Pie, Banana Kush, Wifi, Zkittles, Goji, Sativa Blend, Pineapple Express, Indica Blend, Head Band, Durban Poison, Dog Walker OG, ATF, ACDC, Clementine, Tangie, Sunset Sherbet, Trainwreck, XJ-13, Tahoe OG, Super Lemon Haze, Blue Cheese, Mimosa, Do-si-dos, Sundae Driver, Superglue and Watermelon. FIGS. 1-10 illustrate the terpene profiles/content of some Cannabis strains.

Regarding such films, these films may include strips with the complete terpene content of a Cannabis strain, may include strips with only the major terpenes, or may include terpenes to yield a certain effect, including a certain psychoactive effect such as drowsiness, energy boost, euphoria, relaxation and calming, or a therapeutic effect. Certain terpenes are useful to treat anxiety and depression such as limonene, linalool, and beta caryophyllene. Others are useful as a sleep aid such as myrcene and terpinolene. Others have anti-inflammatory properties such as pinene and beta caryophyllene, and others have anti-tumour properties such as limonene and humulene.

Examples of particular terpene strip combination films having therapeutic utility include a film useful to treat gastrointestinal discomfort comprising limonene, beta caryophyllene, and optionally, gingerol; a film useful to treat anxiety comprising linalool and myrcene, a film useful to treat depression comprising beta caryophyllene and limonene, a film useful to treat inflammation comprising pinene and beta caryophyllene, a film having anti-cancer utility comprising limonene, humulene and guaiol terpene, a film useful to provide an energy boost comprising pinene and eucalyptol, a calming or anti-stress film comprising linalool and myrcene, terpineol, borneol or nerolidol, a sleep-inducing film comprising terpinolene and myrcene, or a pre- or post work-out strip comprising pinene and eucalyptol, and optionally beta caryophyllene, linalool or myrcene. As set out previously, the strips of a film may each include single terpenes, or may include 2 or more terpenes per strip, or any combination thereof.

The films may also comprise one or more terpene-containing strips and one or more strips comprising another active ingredient that complements the terpene content. Thus, the strips of a film may comprise complementary anti-inflammatory treatments (e.g. NSAID or acetaminophen with pinene and/or beta caryophyllene); complementary psychological treatments (e.g. anti-depressive and anti-anxiety agents) with terpenes such as linalool and myrcene (anti-anxiety) or beta caryophyllene and limonene (anti-depression), sleep disorder treatments with terpinolene and/or myrcene; a combination of herbal and/or botanical extracts with selected terpenes such as a combination of limonene, beta caryophyllene, and optionally, gingerol with ginger, sabinene and balsam to treat gastrointestinal discomfort; kana and kava with linalool and myrcene, terpineol, borneol or nerolidol for anti-stress, or with beta caryophyllene and limonene for anti-depression; and the like.

Generally, the present films are made by blending the dry ingredients (e.g. film-forming agents) together and mixing with liquid ingredients including water, other solvents and/or an aqueous phase including water soluble ingredients, to form a homogeneous liquid blend. The film is then extruded/cast and coated onto a moving belt or drum for drying/cutting/rolling. The drying phase may include, for example, air drying, baking, vacuum drying, microwave drying, radio-frequency drying or dehydrating with circulating warm air. Temperature for film extrusion is in the range of about 20-50° C. to produce a film having a moisture level in the range of about 8.5-10%, preferably about 9%. Drying conditions include a temperature of about 40-105° C. and a relative humidity of about 10-60%.

As one of skill in the art will appreciate, slot extrusion techniques are useful to achieve uniform extrusion of the film, and may also be adapted to extrude different films adjacent to one another to yield a film comprising two or more distinct adjacent conjoined strips. In this regard, multiple extruders and specially constructed dies may be useful as described in US 2004/0096569. Generally, a film-forming slurry is fed into a hopper that channels the slurry for extrusion through a selected arrangement of extruder(s) and dies to achieve the desired conjoined strip-containing film.

In one embodiment, the dry ingredients (such as film-forming agents, plasticizer and active ingredients) are blended together, combined with potable water that has a total dissolved solid of lower than 300 part per million, and mixed with a high shear mixer (at about 45-55 rpm) until blended (for 10-60 minutes) at a temperature of about 65-80° C. Liquid ingredients are then added (such as surfactant/emulsifier and flavor agent) and high shear mixing is continued for 10-60 minutes at about 45-55 rpm to form a homogeneous blend. Once mixing is complete, the resulting slurry is allowed to rest for a period of time such as about two hours. Following resting, the slurry is then extruded/cast onto a moving belt, drum or mylar web which passes through a series of heating (about 40-105° C.) and cooling cycles to result in a film with a 8.5-9.5% homogeneous moisture level, preferably about 9%, and a thickness of about 0.001″ to 0.020″ (e.g. 25 to 635 microns).

The present films provide several advantages. For example, the present films readily permit the preparation of an orally administrable product comprising a selected combination of active ingredients, including one or more terpenes with or without one or more additional pharmaceutical, neutraceutical and/or therapeutic agents, and the flexibility to be able to readily alter the film to exclude one or more ingredients, or add additional ingredients. The present film comprising distinct strips also provides a means to deliver two or more active ingredients in a single product while avoiding the potential for any undesirable interaction between the active ingredients during production or post-production. Further, the method of making such films enables the provision of a product containing terpenes and other active ingredients of high quality and in precise quantities. The present films are also adaptable with respect to the delivery of their terpene and other actives due to the presence of distinct strips that may have different dissolution properties to permit staged delivery of the terpenes and any other actives therein. The present films, thus, provide a custom product for delivery of terpenes in a cost effective and efficient manner.

Further, the present films provide a means by which terpenes from non-Cannabis sources can be provided for administration/consumption. This is advantageous since, due to variation in plants and growing seasons, it is difficult to naturally produce consistently a uniform product. The present film product enables the use of terpenes extracted from a vast array of plants to provide a product that possesses a consistent terpene makeup that mimics the content of various cannabis strains, or which is altered from such strains as desired, e.g. with or without CBD and or THC. The present film, thus, provides a cost effective alternative to cultivating Cannabis, since terpene extraction from non-Cannabis plants is accomplished at reduced cost.

In another aspect of the invention, a film is provided comprising a coating of a buccal and/or sublingual mucosal absorption enhancer. A buccal and/or sublingual mucosal absorption enhancer is a compound that functions to increase absorption of the film and its content by stimulating buccal and/or sublingual mucosal absorption. Examples of suitable such absorption enhancers include, but are not limited to, dextro-limonene, nerolidol, alpha-bisabolol, eucalyptol, 23-lauryl ether, aprotinin, atone, taurine, dextran sulfate, cetylpyridinium chloride, lauric acid, menthol, xylitol, oleic acid, polyoxyethylene, polysorbate 80, salts of EDTA, deoxycholate, glycocholate, lauryl sulfate, glycodeoxycholate, salicylate, taurocholate and dodecyl sulphate (e.g. sodium salts), palmitoyl carnitine and alkyl glycosides. For application to a film, the absorption enhancer is encapsulated in a charged powder of maltodextrin, inulin, α-tocopherol, acacia gum, or a mixtures thereof in an amount in the range of about 0.001-5% by wt. The powder is charged by exposure to a high voltage low amperage charge (e.g. 60 kV to 100 kV/25 to 30 microamperes (μA)). The buccal/sublingual absorption enhancer is applied in the charged powder to both sides of the film following its manufacture at a thickness in the range of about 25-200 microns and compressed into the film, for example, by passage between two heated rollers (e.g. heated to about 50 to 98° C.).

In a further aspect, a film is provided comprising an anti-microbial coating, e.g. anti-bacterial, anti-fungal, anti-parasitic coating. The coating may be applied to the film in a manner similar to a buccal/sublingual absorption enhancer coating. Examples of anti-microbial agents that may be coated on the film include, but are not limited to, terpenes such as nerolidol, ocimene, para-cymene, limonene, eucalyptol, citrol, gamma terpinene and geraniol; however, other anti-microbial agents may also be used such as acetic acid, lactic acid, propionic acid, sorbic acid and benzoic acid. The amount of anti-microbial in the powdered coating is in the range of about 0.001-5% by wt, which is applied to the film at a thickness in the range of about 25-200 microns. Preferably the film is coated with nerolidol which has the dual function of buccal absorption enhancer and anti-microbial. The provision of a film with an anti-microbial coating prevents undesirable infection by a user. Since the present films are generally contained in a case, in order to consume the film, it must be removed from the case using fingers, which are not necessarily clean at all times, and then placed in the mouth, providing the opportunity for infection to a user. In some instances, the terpene within the film may function as both the active ingredient and protective anti-microbial agent.

Embodiments of the invention are described in the following specific examples which are not to be construed as limiting.

### Example 1—an Edible Film Having Quick and Slow Dissolving Adjoined Strips

A terpene-containing film comprising 2 adjacent adjoined film strips, a quick dissolving film strip and a slow dissolving film strip, was prepared.

The quick dissolving film was prepared by blending the following dry ingredients: sorbitol (7.3 g/16.73 wt %), sodium alginate (12 g/27.5 wt %) and pullulan (19 g/43.55 wt %). Potable water (100 g) was added to the dry ingredients at 68° C. and mixed with a high shear mixer for 30 min. to form a slurry. Glycerin (1.45 g/3.32 wt %) was added to the slurry which was continuously blended at 50 rpm for 25 min. Limonene was also added to the slurry in an amount of 2.5 g/6 wt %. Once blended, the slurry was allowed to rest for 2 hours.

The slow dissolving film was prepared by blending the following dry ingredients: sorbitol (7.3 g/19.16 wt %), hydroxypropyl methylcellulose (12 g/31.49 wt %), carrageen (5 g/13.12 wt %) and maltodextrin (9 g/23.62 wt %). Potable water (100 g) was added to the dry ingredients at 68° C. and mixed with a high shear mixer for 30 min. to form a slurry. Glycerin (1.45 g/3.81 wt %) was added to the slurry which was continuously blended at 50 rpm for 25 min. Humulene was also added to the slurry in an amount of 2.5 g/6 wt %. Once blended, the slurry was allowed to rest for 2 hours.

The resulting slurries were prepared into a film by being drawn through an extruder adapted to extrude the 2 films adjacently and conjoined onto a moving mylar web which was passed through a series of heating (45° C. to 95° C.) and cooling cycles to obtain a film having a 9% homogeneous moisture level, a quick dissolving terpene strip and a slower dissolving terpene strip.

The quick-dissolving strip was determined to have a dissolve rate of 10 seconds and the slow-dissolving strip had a dissolve rate of 65 seconds utilizing EI QC 019.

### Example 2—an Edible Film Having a Terpene and Plant Extract Adjoined Strips

A film comprising 2 adjacent adjoined film strips, an oregano-containing strip and an alpha bisabolol-containing strip, was prepared.

The film was prepared as above by combining dry ingredients: sorbitol (7.3 g/15.60% by wt), starch (12 g/25.64% by wt), sodium alginate (10 g/21.37% by wt), pullulan (9 g/19.23% by wt), sorbitan monostearate (1.45 g/3.10% by wt) and polysorbate 60 (0.85 g/1.82% by wt). The dry blend was mixed with water (100 g) as previously described to form a slurry which was divided.

Oregano (1.7 g/3.6% by wt) and alpha bisabolol (0.28 g/0.6% by wt) were added to separate slurries pre-extrusion, blended and then rested for 2 hours.

The resulting slurries were prepared into a film by being drawn through an extruder adapted to extrude the 2 films adjacently and conjoined onto a mylar web which was passed through a series of heating (45° C. to 95° C.) and cooling cycles to obtain a film having a 9% homogeneous moisture level.

### Example 3—an Edible Film Coated with an Anti-Microbial Agent

A quick dissolving film strip was prepared as above by combining the following dry ingredients: sorbitol (7.3 g/15.60% by wt), starch (12 g/25.64% by wt), sodium alginate (10 g/21.37% by wt), pullulan (9 g/19.23% by wt), sorbitan monostearate (1.45 g/3.10% by wt) and polysorbate 60 (0.85 g/1.82% by wt). The dry blend was mixed with water (100 g water) as previously described to form a slurry which was divided. Ocimene (0.400 mg) and limonene (0.450 g) were added to separate slurries pre-extrusion, each were blended and then rested for 2 hours.

The resulting slurries were prepared into a film by being drawn through an extruder adapted to extrude the film onto a mylar web which was passed through a series of heating (45 to 90° C.) and cooling cycles to obtain a film having a 9% homogeneous moisture level.

The film was then coated with a charged antibacterial/antifungal agent (Geraniol) that was infused into a starch powder in an amount of 4.8% by wt, and charged by exposure to a high voltage low amperage charge (e.g. 60 kV to 100 kV/25 to 30 microamperes (μA) for 1.5 s. The anti-microbial charged powder was applied to both sides of the film at a thickness in the range of about 25-200 microns and compressed into the film by passage between two heated rollers (e.g. heated to about 50 to 98 degrees ° C.).

### Example 4—an Edible Strip Having Linalool and Myrcene Adjoined Strips

A film comprising 2 adjacent adjoined film strips, a linalool-containing strip and a myrcene-containing strip, was prepared as above by combining the dry ingredients: sorbitol (7.3 g/15.60% by wt), starch (12 g/25.64% by wt), sodium alginate (10 g/21.37% by wt), pullulan (9 g/19.23% by wt), sorbitan monostearate (1.45 g/3.10% by wt) and polysorbate 60 (0.85 g/1.82% by wt). The dry blend was mixed with water (100 g water) as previously described to form a slurry.

Linalool (1.6 mg/0.36% by wt) and myrcene (0.8 mg/0.18% by wt) were added to separate slurries pre-extrusion, blended and then rested for 2 hours.

The resulting slurries are prepared into a film by being drawn through an extruder adapted to extrude the 2 films adjacently and conjoined onto a mylar web which was passed through a series of heating (45 to 90° C.) and cooling cycles to obtain a film having a 9% homogeneous moisture level.

### Example 5 an Edible Film Coated with a Buccal/Sublingual Mucosal Absorption Enhancer

A quick dissolving film strip was prepared as above by combining the following dry ingredients: sorbitol (7.3 g/15.60% by wt), starch (12 g/25.64% by wt), sodium alginate (10 g/21.37% by wt), pullulan (9 g/19.23% by wt), sorbitan monostearate (1.45 g/3.10% by wt) and polysorbate 60 (0.85 g/1.82% by wt). The dry blend was mixed with potable water (100 g water) as previously described to form a slurry. Ocimene (0.400 mg) and eucalyptol (0.450 g) were separately added to slurry pre-extrusion, each were blended and then rested for 2 hours

The resulting slurries were prepared into a film by being drawn through an extruder adapted to extrude the film onto a moving belt, drum or mylar web, which was then passed through a series of heating (45 to 90° C.) and cooling cycles to obtain a film having a 9% homogeneous moisture level.

The film was then coated with a charged buccal/sublingual absorption enhancer (limonene) that was infused into a starch powder in an amount of 4.8% by wt, and charged by exposure to a high voltage low amperage charge (e.g. 60 kV to 100 kV/25 to 30 microamperes (μA) for 1.5 s. The buccal absorption enhancer charged powder was applied to both sides of the film at a thickness in the range of about 25-200 microns and compressed into the film by passage between two heated rollers (e.g. heated to about 50 to 98 degrees ° C.).

The film was then coated with a charged buccal absorption enhancer (limonene) that was infused into a starch powder in an amount of 4.8% by wt, and charged by exposure to a high voltage low amperage charge (e.g. 60 kV to 100 kV/25 to 30 microamperes (μA) for 1.5 s). The buccal absorption enhancer charged powder was applied to both sides of the film at a thickness in the range of about 25-200 microns and compressed into the film by passage between two heated rollers (e.g. heated to about 50 to 98 degrees ° C.).

1. An edible film comprising two or more selected terpenes from non-Cannabis sources, at least one film-forming agent, at least one surfactant or emulsifier, and a plasticizer, wherein the film comprises at least two adjacent adjoined strips, each strip comprising a different selected terpene or combination of terpenes, wherein each terpene is present in a quantity ranging from about 0.01% to 30% by wt of the film.

2. The edible film of claim 1, wherein each terpene is selected from the group consisting of a hemiterpenes, monoterpenes, sesquiterpenes, diterpenes, sesterterpenes, triterpenes, sesquarterpenes, tetraterpenes, hydroxylated terpenes, oxygenated terpenes, and mixtures thereof.

3. The edible film of claim 1, wherein each terpene is selected from the group consisting of: myrcene, beta caryophyllene, pinene, limonene, terpinolene, humulene, nerolidol, linalool, ocimene, guaiol, bisabolol, alpha phellandrene, cadinene, camphene, camphor, citral, citronellol, delta 3-carene, eucalyptol, eugenol, gamma terpinene, geraniol, humulene, nerol, nerolidol, ocimene, paracymene, phytol, pulegone, terpineol, valencene, prenol, isovaleric acid, geraniol, an iridoid, farnesene, farnesol, B-bisabolene, B-curcimene, lanceol, juvabione, humbertiol, cafestol, kahweol, cembrene, taxadiene, geranylfarnesol, squalene, ferrugicadiol, tetraprenylcurcumene, acyclic lycopene, monocyclic gamma-carotene, bicyclic alpha- or beta-carotene, oxides thereof, hydroxylated analogues thereof and mixtures thereof.

4. (canceled)

5. The edible film of claim 1, comprising a film forming agent in an amount ranging from 30 to about 80 wt %, a surfactant or an emulsifier in an amount ranging from 0.5 to 15% by wt, and a plasticizer in an amount ranging from 2 to 18% by wt.

6. The edible film of claim 1, wherein the film forming agent is selected from the group consisting of pullulan, sodium alginate, carrageenan, methyl cellulose, ethyl cellulose, hydroxypropylmethyl cellulose, hydroxypropyl cellulose, corn starch, pectin, chitin, chitosan, collagen, gelatin, zein, gluten, cassava starch, glucan and mixtures thereof.

7. The edible film of claim 6, additionally comprising one or more agents that facilitate film forming selected from the group consisting of hydroxyethyl cellulose, polyvinyl pyrrolidone, carboxymethyl cellulose, polyvinyl alcohol, polyethylene glycol, polyacrylamides, polyvinyl alcohol, polyethylene glycol, glycolide, polylactic acid, polylactide, cellulose gum, xanthan gum, tragacanth gum, guar gum, acacia gum, arabic gum, polyacrylic acid, sodium methacrylic acid polymers, methacrylic acid copolymers, acrylic acid polymers, acrylic acid copolymers, methylmethacrylate copolymer, carboxyvinyl polymer, amylase, high amylose starch, casein, algin, maize, chondrus crispus, crosspovidone (CPV), polyvinylpyrrolidone (PVP), hydroxymethylcellulose, gum tragacanth, gum karaya, glycogen, phytoglycogen, welan gum, polysaccharides, hydroxypropylate, dextrin, maltodextrin, soy protein isolate, whey protein isolate, beeswax, carnauba wax, fungal exopolysaccharides and rapeseed.

8. The edible film of claim 1, comprising a surfactant selected from the group consisting of glycerol, glycerin, propylene glycol, sucrose fatty acid ester, sorbitan monostearate, emulsifier comprising sorbitol, lecithin, modified cellulose gum (CMC), ceratonia gum, locust bean gum, sodium lauryl sulfate (SLS) and mixtures thereof.

9. The edible film of claim 1, comprising an emulsifier selected from the group consisting of poly-oxyethylene, sorbitan monostearate, lecithin, stearate salt, calcium stearoyl lactylate (CSL), glyceryl monostearate, mono propyleneglycol, sorbitan monooleate, emulsifier comprising sorbitol, monoglyceride, glycerol triacetate and mixtures thereof.

10. The edible film of claim 1, comprising a plasticizer selected from the group consisting of monostearate palm oil, lethicin, sorbitan monostearate, glycerin, glycerin fatty acid ester, triacetin, pre-gelatinized starch, sorbitol, a medium chain mono- or diglyceride, castor oil, malic acid, polyethylene glycol (PEG), glycerol, palm oil and mixtures thereof.

11. The edible film of claim 1, additionally comprising a sweetener and/or a flavouring agent.

12. The edible film of claim 1, comprising an additional active ingredient is a nutraceutical or a pharmaceutical agent in an amount in the range of 0.01% to 15% by wt.

13. The edible film of claim 1, wherein each strip exhibits a different rate of dissolution.

14. The edible film of claim 1, comprising a first strip made of a film forming agent selected from pullulan, sodium alginate, and mixtures thereof, and a second strip made of a film forming agent selected from hydroxypropyl methylcellulose, carrageen, and mixtures thereof

15. The edible film of claim 1, additionally comprising a buccal and/or sublingual mucosal absorption enhancer.

16. The edible film of claim 15, wherein the buccal and/or sublingual mucosal absorption enhancer is selected from 23-lauryl ether, aprotinin, 1-dodecylazacycloheptan-2-one, taurine, dextran sulfate, cetylpyridinium chloride, lauric acid, menthol, xylitol, oleic acid, polyoxyethylene, polysorbate 60 or 80, salts of EDTA, deoxycholate, glycocholate, lauryl sulfate, glycodeoxycholate, salicylate, taurocholate or dodecyl sulphate, palmitoyl carnitine, an alkyl glycoside, alpha-limonene, nerolidol, limolene and mixtures thereof.

17. The edible film of claim 1, comprising an anti-oxidant.

18. The edible film of claim 17, wherein the antioxidant is selected from sodium thiosulfate, ascorbic acid, fatty acid esters of ascorbic acid, alpha-tocopherol, propyl gallate, erythorbic acid, sodium erythorbate, citric acid, tocopherols, alkyl-substituted paraben or salts thereof, and mixtures thereof.

19. The edible film of claim 1, coated with a buccal and/or sublingual mucosal absorption enhancer.

20. The edible film of claim 1, coated with an anti-microbial agent.

21. The edible film of claim 1, wherein the film is coated with a buccal and/or sublingual mucosal absorption enhancer or an anti-microbial agent or both encapsulated in a charged powder.

22. The edible film of claim 21, wherein the buccal and/or sublingual mucosal absorption enhancer is selected from 23-lauryl ether, aprotinin, azone, taurine, dextran sulfate, cetylpyridinium chloride, lauric acid, menthol, xylitol, oleic acid, polyoxyethylene, polysorbate 60 or 80, salts of EDTA, deoxycholate, glycocholate, lauryl sulfate, glycodeoxycholate, salicylate, taurocholate or dodecyl sulphate, palmitoyl carnitine, an alkyl glycoside, alpha-limonene, nerolidol, limolene and mixtures thereof.

23. The edible film of claim 21, wherein the buccal and/or sublingual absorption enhancer is encapsulated in a charged powder of maltodextrin, inulin, α-tocopherol, acacia gum, or a mixtures thereof.

24. The edible film of claim 21, wherein the anti-microbial agent is an anti-bacterial agent, an anti-fungal agent, an anti-viral agent, an anti-parasitic agent, or a mixture thereof.

25. The edible film of claim 21, wherein the anti-microbial agent is selected from nerolidol, ocimene, para-cymene, limonene, eucalyptol, citrol, gamma terpinene, geraniol and mixtures thereof.

26. The edible film of claim 21, wherein the anti-microbial agent is applied as coating which is 25-200 microns thick and comprising 0.001-5% by wt of the anti-microbial agent.

27. (canceled)

28. The edible film of claim 1, comprising 2 or more terpenes that mimic the terpene content of a Cannabis plant.

29. The edible film of claim 1, comprising a cannabinoid.
